(19) United States
(12) Patent Application Publication
(10) Pub. No.: US 2003/0208785 A1
(43) Pub. Date: Nov. 6, 2003

(54) Transgenic pig containing heat shock protein 70 transgene

(75) Inventors: Bor-Show Tzang, Miaoli Hsien (TW); Ching-Fu Tu, Miaoli Hsien (TW); Wen-Chuan Lee, Miaoli Hsien (TW); Ming-Yu Chen, Miaoli Hsien (TW); San-Yuan Huang, Miaoli Hsien (TW); Jyh-Hung Lin, Miaoli Hsien (TW)

Correspondence Address: ROTHWELL, FIGG, ERNST & MANBECK, P.C., 1425 K STREET, N.W., SUITE 800, WASHINGTON, DC, 20005, US

(73) Assignee: Animal Technology Institute Taiwan, Miaoli Hsien (TW)

(21) Appl. No.: 10/138,317

(22) Filed: May 6, 2002

(51) Int. Cl. A01K 67/027; C07H 21/04

(52) U.S. Cl. 800/17; 536/23.5

(57) ABSTRACT

The invention provides a transgenic pig having incorporated into its genome a HSP70 gene or the fragment thereof, whereby said transgenic pig overexpresses HSP70. The transgenic pig of the invention can be used in the production of HSP in large quantity and used as a xenograft source for transplation and an animal model close to human for illustrating the protective roles of HSP. Furthermore, the transgenic pig of the invention has a better meat quality and exhibits an increased growth rate and a reduced backfat thickness.

### BACKGROUND OF THE INVENTION

1. Field of the Invention

The present invention relates to a transgenic pig for overexpressing heat shock protein 70.

2. Description of the Prior Art

When exposed to nonlethal heat shock, a variety of organisms and cell ones acquire transient resistance to subsequent exposures to elevated temperatures. This phenomenon has been termed thermotolerance. Heat shock protein (hereafter HSP) 70 has been described playing an important role in thermoresistance. HSP70 also is closely related to food intake, growth rate and backfat thickness of pigs. The HSP is synthesized in cells in response to an increase of temperature above normal physiological levels, or following exposure to a variety of toxic agents. Recently, elevated expression of HSP70 and other HSPs has been observed in cells and tissues under conditions representative of human diseases, including ischemia, oxidant injury, immunology and infectious diseases (Marber, et al., 1995, J. Clin. Invest. 95:1446-1456 and Kiang et al., 1998, Pharmacol. Ther. 80: 183-201). The increased expression of these stress proteins could represent an acute response to altere physiological states, as well as chronic adaption to some diseases. The primary function of these stress responses is thought to be cytoprotective. For example, overexpression of HSP70 alone was demonstrated to protect cells from thermal injury and to increase cell survival. By the overexpression of rat inducible HSP70, the postimplantation murine embryos were protected from heat treatment (Mirkes et al., 1999, Developmental Dynamics, 214:159-170). The protective role of HSP70 was clearly demonstrated by recent studies with transgenic mice in which overexpression of human or rat inducible HSP70 protected myocardium from ischemia reperfusion injury (Marber, et al., 1995, J. Clin. Invest. 95:1446-1456; and Plumier, et al., 1995, J. Clin. Invest., 95:1854-1860).

Most studies concerning HSP70 were performed in in vitro cell or mice or rat models. However, since the in vitro model and the rat model are far from the human, they cannot be successfully applied in human. There is still a need to develop an animal model close to human for developing the effect of the HSP.

### SUMMARY OF THE INVENTION

One object of the invention is to provide a transgenic pig incorporated into its genome an HSP70 gene or the fragment thereof, wherein said transgenic pig overexpresses HSP70.

A further object of the invention is to provide a transgene containing a pig HSP70 gene as shown in SEQ ID NO:1 or the fragments thereof and an expression vector containing the transgene.

### BRIEF DESCRIPTION OF THE DRAWINGS

FIG. 1 shows the map of the hCMV/HSP70.2-GFP transgene.

FIG. 2 shows the intracellular distribution of the porcine HSP70.2-GFP expressed in monkey COS-7 cells analyzed by direct fluorescence.

FIG. 3 shows the western blot of samples prepared from tail clips.

The samples are probed with a polyclonal anti-HSP70 antibody (A) and a monoclonal anti-GFP antibody and anti-actin antibody (B).

FIG. 4 shows the green fluorescence performed in the cultured primary fibroblast cells taken from transgenic pigs' ear.

FIG. 5 shows cell survival after heat shock wherein D-01 represents non-transgenic pig and D-11 and D-12 represent transgenic pig.

### DETAILED DESCRIPTION OF THE INVENTION

The present invention features a transgenic pig having incorporated into its genome an HSP70 gene or the fragment thereof. The invention successfully constructs transgenic pigs stably expressing an HSP70 gene. The transgenic pig of the invention can be used as an animal model close to human for realizing the protective roles and the clinical importance of HSP. Moreover, the transgenic pig of the invention provides the organ sources with low rejection for transplanting.

Definitions

The term “heat shock protein 70 gene (HSP70 gene)”, as used herein, refers to a multigene family with molecule masses of about 70 kDa. The HSP70 gene not only can be induced by heat but also amino acid analogs, glucose analogs, heavy metals, protein kinase C stimulators, Ca++-increasing agents, ischemia, sodium arsenite, microbial infections, nitric oxide, hormones and antibiotics.

The phrase “incorporated into its genome”, as used herein, refers to pigs or other mammals which have a selected transgene introduced into their germ cells and/or somatic cells such that it is stably incorporated and is capable of carrying out a desired function.

The term “genome”, as used herein, refers to the entire DNA complement of an organism, including the nuclear DNA component, chromosomal or extrachromosomal DNA, as well as the cytoplasmic domain (e.g., mitochondrial DNA).

The term “transgene”, as used herein, refers to the introduction of a desired DNA sequence into the animal's genome, including but not limited to genes or DNA sequences which may not normally be present in the genome, genes which are present, but not normally transcribed and translated (“expressed”) in a given genome, or any other genes or DNA sequences which one desires to introduce into the genome. This may include genes which may normally be present in the nontransgenic genome but which one desires to have altered in expression, or which one desires to introduce in an altered or variant form.

The term “expression vector”, as used herein, refers to a vector capable of directing the expression of a gene to which it is operatively linked. In general, an expression vector of utility in recombinant DNA techniques is often in the form of a “plasmid” which refers generally to a circular double stranded DNA loop which, in its vector form, is not bound to the chromosome.

Transgene and Vector System

Transgene are constructed for introducing into the germ line of an animal to make a transgenic pig. According to the invention, any suitable HSP70 gene can be used to be incorporated into the genome of pigs. Preferably, said HSP70 gene is HSP70.2 gene. Preferably, said HSP70 gene is obtained from human or pig. More preferably, said HSP70 gene is selected from the group consisting of pig HSP70.2 gene and human HSP70.2 gene.

In one preferred embodiment of the invention, a new cloned pig HSP70 gene (SEQ ID NO:1) is used to be incorporated into the genome of pigs. The new gene is cloned from pig testis cDNA and has 1923 nucleic acids.

According to the invention, any suitable vector system can be used to produce the transgenic HSP gene for being incorporated into the genome of pigs. Methods well known to those skilled in the art may be used to construct expression vectors containing the HSP gene and appropriate transcriptional and translational control elements. These methods include in vitro recombinant DNA techniques, synthetic techniques, and in vivo genetic recombination.

Transgenic Pig

One object of the invention is to provide a transgenic pig having incorporated into its genome an HSP70 gene or the fragment thereof, wherein said transgenic pig overexpresses HSP70.

According to the invention, transgenic pigs are produced by introducing transgenes into the germline of the pig. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness.

Introduction of the transgene into the embryo can be accomplished by any means known in the art such as, for example, microinjection, electroporation, or lipofection. For example, the HSP70 transgene can be introduced into a mammal by microinjection of the construct into the pronuclei of the fertilized mammalian egg(s) to cause one or more copies of the construct to be retained in the cells of the developing mammal(s). Following the introduction of the transgene construct into the fertilized egg, the egg may be incubated in vitro for varying amounts of time, or reimplanted into the surrogate host, or both. In vitro incubation to maturity is within the scope of this invention.

Transgenic offspring may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful in detecting the presence of the transgene product in the blood, as well as in evaluating the effect of the transgene on the levels of various types of blood cells and other blood constituents.

Utility

It is known that HSPs can be detected in all cells, prokaryotic and eukaryotic. Cells or multi-cell organisms respond to heat or other stresses by inducing or increasing the synthesis of HSPs. Increased levels of HSPs occur after heat stress, environmental stresses, infection, normal physiological processes and gene transfer. Based on the ability of HSPs to protect cells against various stressors, the transgenic pig of the invention highly expressing the HSPs has widely applications.

According to the invention, the transgenic pig of the invention can be used to detect environmental stressors associated with the production of HSPs. Previous studies indicated that transition series metals and sulfhydryl reagents induce the synthesis of HSP70 (Levinson et al., Biochimica et Biophysica Acta, 1980, 606, 170-180; Johnston et al., The Journal of Biological Chemistry, 1980, Vol. 255, No. 14, pp. 6975-6980). Thus, The environmental stressors such as air, land or heavy metal pollution can be detected by assaying the production of HSPs in the transgenic pig of the invention.

According to the invention, the transgenic pig of the invention also can be used as an animal model close to human for illustrating the function of HSP in increasing cell survival and protecting cells from thermal injury, such as sepsis, ischemia reperfusion injury, oxidant injury, atherosclerosis and aging.

According to the invention, the transgenic pig may be used as a xenograft source such as xenograft organs, tissues or cells for transplantation. The xenograft of the invention can reduce the rejection after transplantation, including a xenograft-organ such as kidney, liver, heart, pancreas and lung; a xenograft-tissue such as skin, intestines, endocrine glands, islets, stem cells bone marrow and vascular tissue; and a xenograft-cell such as embryo cell, sperm cell and ovum cell.

According to the invention, the transgenic pigs permit the production of HSPs in a large quantity, which is easily recoverable. For instance, HSP can be produced in the mammary gland of transgenic pigs and excreted in their milk. It is indeed a biological fluid that can be easily collected, having a relatively limited complexity and a low proteolytic activity. Given the above, HSP having a biological activity can be mass-produced from the transgenic pig of the invention.

According to the invention, the transgenic pig has an improved meat quality and improved performance on growth and reproduction for the reason that the transgenic pig has an elevated expression of HSP, which can be against heat stress. It is known that the growth, reproductive performance, and meat quality of pigs will be reduced when they are under heat stress. For example, Becker et al. indicated that the pigs under heat stress reduce the growth performance and their meat quality (Becker et al., J. Anim. Sci. 71:2375-2387). Wettemann et al. described that the influence of elevated temperature on reproductive performance of boars (Wettemann et al., J. Anim. Sci. 42:664-669). Thus, the transgenic pig of the invention overexpressing HSP exhibits a superior growth, such as increased food intake and growth rate, a low backfat thickness, a better meat quality, and an increased reproductive performance.

The following examples are offered by way of illustration and not by way of limitation.

### EXAMPLES

### Example 1

### Production of Transgenic Pig of the Invention

The transgenic pigs of the invention were generated using a chimeric transgene consisting of a porcine inducible HSP70.2 gene inserted into the vector pcDNA3.1/CT-GFP-TOP.

Preparation of the DNA Fragments for Microinjection

The porcine HSP70.2 gene was prepared from testis cDNA by reverse transcription-PCR with the following primers:

| | | |
|---|---|---|
| F5: | 5′-AACATGGCGAAGAGCGTGGCC-3′ | (SEQ ID NO:2) |
| R8: | 5′-CCACCTCCTCGATGGTGGGG-3′ | (SEQ ID NO:3) |

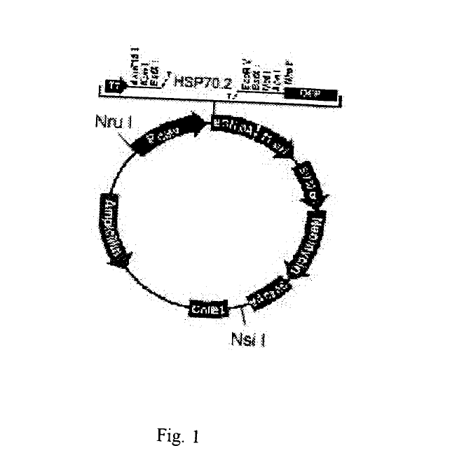

The HSP70 expression vector was constructed using CT-GFP Fusion TOPO Cloning Kit (Invitrogen, Carlsbad, Calif., USA), in accordance with the manufacturer's instructions. Each TOPO cloning reaction contains 100 ng of PCR product, 1 μl of pcDNA3.1/CT-GFP-TOPO, and judged total volume to 5 μl by sterile water. After the resulting solution was gently mixed and incubated for 5 minutes, 1 μl of the 6× TOPO Cloning Stop Solution was added immediately and then the solution was mixed for about 10 seconds at room temperature. A vial of One Shot cells was added to 2 μl of the TOPO Cloning, and they were then mixed gently. After being incubated on ice for 30 minutes, the cells were heat shocked at 42° C. for 30 seconds without shaking. The cells were immediately transferred to ice and incubated for 2 minutes. 250 μl of SOC medium under room temperature was added to the cells. 10 to 50 μl of solution from each transformation were spread on a prewarmed LB plate containing 50 μg/ml ampicillin and incubated overnight at 37° C. The pcDNA3.1/CT-GFP-TOPO construct places the HSP70.2 gene under the control of the human cytomegalovirus immediately enhancer and promoter. FIG. 1 shows the map of the hCMV/HSP70.2-GFP transgene. The coding region of the porcine inducible HSP70.2 gene and GFP gene is under the control of the human CMV immediate-early (hCMV-IE) enhancer and promoter. The chimeric gene is followed by the SV40 polyadenylation signal. The Nru I to Nsi I fragment was used to generate transgenic pigs. The chimeric transgene was cut out of the plasmid by Nru I and Nsi I digestion, purified, and used to generate transgenic pigs. The cut fragments were dissolved in 10 mM Tris-HCl, pH 7.5, 0.1 mM EDTA to a final concentration of 2-4 μg/ml.

Animals and Treatment

Pure breed Duroc gilts being at least seven and half months old were used. The animals were fed with 1.0 to 1.2 kg of commercial feed twice per day and water ad libitium. Sows in lactation were fed with lactation feed and water ad libitium. The transgenic piglets were weaned at 28 to 56 days.

All embryos donor and recipient gilts were synchronized by feeding Regumate (containing 0.4% altrenogest; 20 mg/day; Intervet, Boxmeer, Netherlands) mixed with commercial feed in the morning for 15 days, superovulated by injection of PMSG (1500-2000 IU, i.m., China Chem. And Pharm., Taiwan) at 24 hrs after the last feeding Regumate and injection of hCG (1250-1750 IU, i.m., China Chem. And Pharm., Taiwan) at 76 to 78 hrs after the injection of PMSG, and mated by artificial insemination with pure breed Duroc boars' fresh-diluted semen at 24 to 36 hrs after the injection of hCG.

At 54 to 56 hrs after the injection of hCG, the donor pigs were to surgically operated to flush fertilized eggs from the fallopian tubes with 20 ml Dubacos-PBS (Gibco/BRL, USA) with 0.4% BSA (Fraction V, Sigma, USA) into a dish. Before operation, pigs were fasted overnight, and were calned by injection (i.m.) with 5 ml sterinil (2 mg/kg, Janssen Pharmaceutical, Belgium) and 10 ml atropine sulfate (90.04 mg/kg, China Chem. And Pharm., Taiwan). Then, they were initially anaesthetized by injection of sodium pentobarbitone (10 mg/kg, Abbott Australasia Pty Ltd., Australia) into an ear vein. Anesthesia was maintained throughout the operation via a closed-circuit system using 4% halothene (ICI Ltd., USA) inhalation with oxygen gas.

Production of Transgenic Pigs

The DNAs used for injection were diluted with TE buffer (10 mM Tris-HCL, 0.1 mM EDTA, pH 7.4) to 2 ng/μl for HSP70 clone.

The fertilized eggs were centrifuged with 23,500×g for 8 min in room temperature by centrifuge (Hettich EBA 12, Germany) to expose pronuclei. The pig embryos were micromanipulated by Leica mechanical manipulator with differential interference contrast inverted microscope (ZEISS Axiovert 135, Germany). The transgene was injected to the pronucleus of new fertilized pig eggs or nuclear of two-cell of pig embryos. After about 25 to 30 pig embryos were injected, the embryos were transferred into the fallopian tubes of recipient-synchronized as soon as possible.

Analysis of Transgene

After pregnancy of sow was complete and piglets were delivered, the ear tissue of live piglets or tail tissues of the stillborn piglets were taken to extract genomic DNAs at the delivery day.

The transgenes were screened by PCR with primers:

| | |
|---|---|
| F8: | |
| 5′-GACGCCAACGGCATCCTGAAC-3′ | (SEQ ID NO:4) |
| GFP-reverse: | |
| 5′-TAGAAGGCACAGTCGAGG-3′ | (SEQ ID NO:5) |

The PCR reaction was conducted as follows:

The reaction products were analyzed by 2% agarose gel-electrophoresis.

As shown in Table 1, there were 2 piglets carried the transgene examined by PCR using primers F8 and GFP-reverse. The transfer rate was 16.7%.

TABLE 1

| No. of embryo | No. (%) of foster | | No. (%) of pup | |
|---|---|---|---|---|
| Microinjected | Transferred | Delivered | Born/Analyzed | Tg |
| 181 | 8 | 4(50) | 12/12 | 2(16.7) |

### Example 2

### Analysis of HSP of the Transgenic Pig

Protein Analysis in Transfected Cell

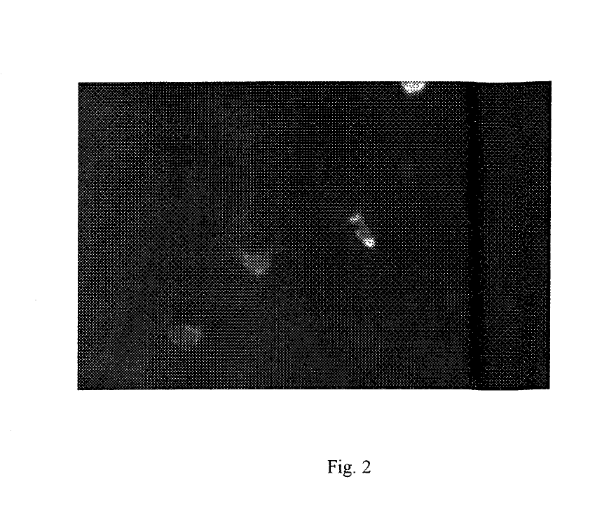

Expression of porcine HSP70.2-encoding gene in transfected COS-7 cells was first verified by observing green fluorescence directly from HSP70.2-GFP fussion protein. Monkey kidney COS-7 cells were grown in Dulbecco's modified medium supplemented with 10% fetal bovine serum and appropriate antibiotics. Exponentially growing COS-7 cells were transfected with a plasmid containing the porcine gene for HSP70.2 using a SuperFect Transfection Reagent (Qiagen, Chatsworth, Calif., USA), in accordance with manufacturer's instructions. Neomycine-resistant cells were selected in medium containing G418 (400 μg/ml), and colonies were isolated, trypsinized, and grown to confluent monolayers for further characterization. FIG. 2 shows the intracellular distribution of the porcine HSP70.2-GFP expressed in monkey COS-7 cells analyzed by direct fluorescence wherein the green fluorescence represents the HSP70.2-GFP fusion protein.

Protein Analysis in Transgenic Pig

Pigs underwent protein analysis to determine the expression of transgenic HSP-GFP. The tails or ears were taken in 3 days and 6 months old and placed in lysis buffer on ice. The tissues were then homogenized using a polytron and placed directly on ice. The homogenized slurry was transferred to a clean tube and centrifuged at 12,000 rpm for 5 min at 4° C. The supernatant was aliguoted to sterile microcentrifuge tubes. The aliquoted samples was kept at −20° C. until used.

Total proteins were electrophoresed in 9% SDS-PAGE transferred by electrophoresis to nitrocellulose filters and reacted with anti-HSP70 polyclonal antibody (Stressgen SPA-812) or with anti-GFP monoclonal antibody (Invitrogen). They were then incubated with an anti-rabbit IgG-alkaline phosphates conjugated secondary antibody. Specific antibody binding was detected using NBT/BCIP.

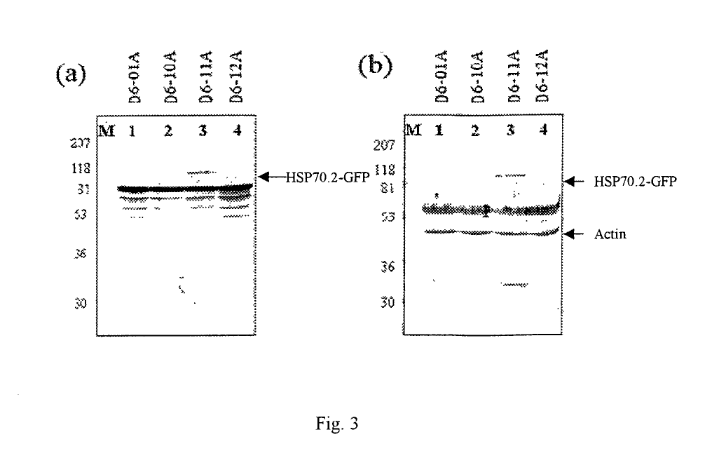

Expression of porcine HSP70.2 in transgenic pigs was confirmed by immunoblot analysis using anti-HSP70 pAb (Stressgen SPA-812, specific against the inducible form). The tissue proteins separated by using 9% SDS-PAGE were detected via the immunoblot analysis. The western blot of samples prepared from tail clips was shown in FIG. 3. The samples are probed with a polyclonal anti-HSP70 antibody (A) and a monoclonal anti-GFP antibody and anti-actin antibody (B). The additional 100-kDa polypeptide is the HSP70.2-GFP fussion protein.

### Example 3

### Viability of Cells with or without HSP70 under Stress

Fibroblast Cell Culture from Piglet's Ear

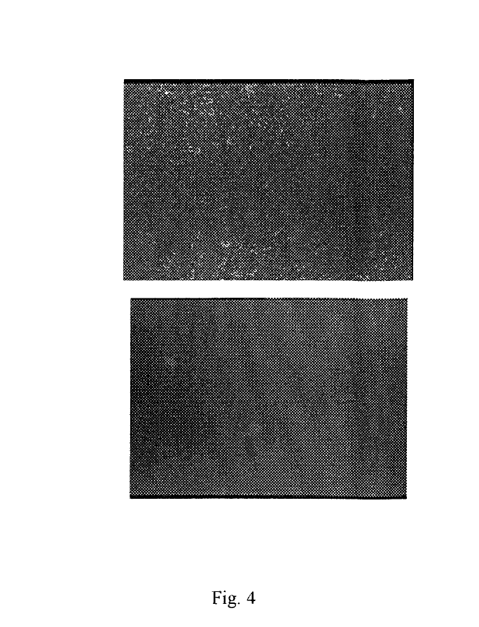

Primary fibroblast isolated from the ears of the transgenic pigs which were seven months old and were cultured. After dispase (Sigma) treatment at 37° C. for 2 hrs, the ear tissues were then immersed in 0.1% collagenase (Sigma) at 37° C. for another 2 hrs. The cells released to the suspension were collected by centrifuging at 800 rpm for 10 min and were subsequently cultured in a 25 mm flask. As shown in FIG. 4, the green fluorescence performed in the cultured fibroblast cells taken from transgenic pigs' ear is HSP70.

Heat Treatment and Cell Survival

The primary fibroblast cells were grown to 90% confluent and then treated with lethal heat shock at 45° C. for 3 hrs in a water bath. Subsequent to this period, the media were removed to a centrifuge tube and the cells released to the media were collected by centrifuging at 800 rpm for 10 min. The cells still attached on the tissue culture plates were combined with those released to media as prior described. The cell viability was assessed by trypan blue exclusion as described by Riabowol et al, 1988, Science, 242 (4877): 433-436. All experiments were done at least three times and yielded consistent results.

The ability of experimental and control primary fibroblasts to survive a lethal heat treatment was examined. The primary fibroblasts of the transgenic pigs and non-transgenic pigs were object to an initial heat treatment. A trypan-blue exclusion assay (Riabowol et al. 1988) and aptitude for colony formation test were used to estimate cell survival.

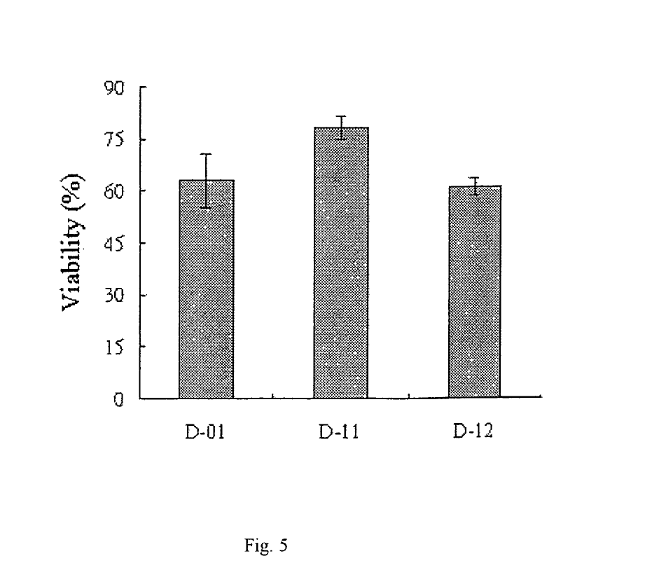

The primary fibroblasts receiving a heat stress at 43° C. for 3 hrs, survive the lethal heat challenge. Up to 75% of cells survive when cultured from transgenic animal overexpressing, whereas 60% of cells survive when cultured from non-transgenic animal (P=0.018). FIG. 5 shows the cell survival after heat shock wherein D-01 represents non-transgenic pig and D-11 and D-12 represent transgenic pigs. The result evidenced that HSP70 protein was at least partially responsible for protection of cells against a lethal heat treatment.

What is claimed is:

1. A transgenic pig having incorporated into its genome a HSP70 gene or the fragment thereof, whereby said transgenic pig overexpresses HSP70.

2. The transgenic pig according to claim 1, wherein said HSP70 gene is HSP70.2 gene.

3. The transgenic pig according to claim 1, wherein said HSP70 gene is obtained from human or pig.

4. The transgenic pig according to claim 1, wherein said HSP70 gene is selected from the group consisting of pig HSP 70.2 gene and human HSP 70.2 gene.

5. The transgenic pig according to claim 1, wherein said HSP70 gene is the pig HSP70.2 gene as shown in SEQ ID NO: 1.

6. The transgenic pig according to claim 1, for use as the source of a xenograft organ, a xenograft tissue or a xenograft cell with low rejection for transplantation.

7. The transgenic pig according to claim 6, wherein said xenograft organ is kidney, liver, heart, pancreas or lung.

8. The transgenic pig according to claim 6, wherein said xenograft tissue is skin, intestines, endocrine glands, islets, stem cells bone marrow or vascular tissue.

9. The transgenic pig according to claim 6, wherein said xenograft cell is embryo cell, sperm cell or ovum cell.

10. The transgenic pig according to claim 1, for use in detecting the environmental stressors associated with the production of HSP.

11. The transgenic pig according to claim 10, wherein the environmental stressor is air, land or heavy metal pollution.

12. The transgenic pig according to claim 1, for use as an animal model close to human for illustrating the function of HSP in protecting cells from thermal injury.

13. The transgenic pig according to claim 12, wherein said thermal injury is sepsis, ischemia reperfusion injury, oxidant injury, atherosclerosis or aging.

14. The transgenic pig according to claim 1, which produces HSPs large quantity in the mammary gland.

15. The transgenic pig according to claim 1, which has a better meat quality and exhibits an increased growth rate and a reduced backfat thickness.

16. The transgenic pig according to claim 1, which exhibits an increased reproduction performance.

17. A transgene, which contains a pig HSP70 gene as shown in SEQ ID NO:1 or the fragments thereof.

18. An expression vector containing the transgene as claimed in claim 17.
